(19) United States
(12) Patent Application Publication
(10) Pub. No.: US 2012/0324783 A1
(43) Pub. Date: Dec. 27, 2012

(54) JATROPHA HYBRIDS THROUGH FEMALE ONLY TRAIT

(75) Inventor: Richard Rotter, Encinitas, CA (US)

(73) Assignee: SG BIOFUELS, LTD., Grand Cayman, KY (KY)

(21) Appl. No.: 13/520,681

(22) PCT Filed: Dec. 30, 2010
(86) PCT No.: PCT/US10/62525

§ 371 Date: Aug. 8, 2012

Related U.S. Application Data
(60) Provisional application No. 61/292,751, filed on Jan. 6, 2010.

(51) Int. Cl. A01H 5/10; A01H 1/02; C10L 1/00; C11B 1/10

(52) U.S. Cl. 44/307; 554/9; 800/260; 800/298

(57) ABSTRACT

Jatropha curcas plants can be produced that are characterized by an inflorescence with female-only (FO) flowers. Such plants are especially useful for interplanting in order to obtain commercial scale production of new J curcas hybrids derived from the seeds of the FO-type plant.

### CROSS-REFERENCE TO RELATED APPLICATION

This application claims the benefit of U.S. Provisional Application No. 61/292,751, filed Jan. 6, 2010, herein incorporated by reference in its entirety.

### FIELD OF TECHNOLOGY

The present invention relates generally to the field of Jatropha crop selection and breeding. In particular, the present invention relates to novel J. curcas plants producing inflorescence with “female only” (FO) flowers, to methodology for production of new J. curcas hybrids using inflorescences with a novel “female only” (FO) flower trait, and to seed and other products obtained from such J. curcas hybrids.

### INTRODUCTION

The present invention relates to the Jatropha plant, which is known botanically as Jatropha curcas of the Euphorbiaceae family and which is commonly referred to as “Physic Nut.” The genus Jatropha includes over 175 species of succulent, perennial trees or shrubs. Jatropha curcas is a drought-resistant, perennial species of Latin American origin, which has become widespread throughout the tropical and subtropical areas of the world, including India, Africa, Asia and North America.

More specifically, J. curcas is a small, diploid (2 n=22) tree or shrub with smooth, gray or reddish bark, which exudes whitish colored, watery, latex when cut. Jatropha curcas typically grows to between three and five meters in height, but it can grow up to eight or ten meters under favorable growing conditions. Jatropha curcas plants have a vigorous growth rate and can produce fruit and seeds for up to 50 years, even when cultivated on marginal lands. Seed of J. curcas plants produce oil, which can be processed for use as a diesel and jet fuel substitute.

The combined characteristics of the J. curcas plant as a non-agronomic crop; being adaptable for cultivation on semi-arid and marginal soil sites; and the high oil content of the produced seeds has initiated interest in crop improvement programs. To date, however, only a limited literature and related genetic information have been available to aid the development of J. curcas plants with desirable traits, such as earlier maturity, early flowering, increased female flowering, increased fruit and seed count, and increased oil yield.

For other cross-pollinated plants, the breeding and selection process of hybridization has been employed to modify or to improve traits, efficiently and economically. Maximum genetic uniformity and improved vigor and yield in hybrids is obtained by utilizing two distinct parental plant populations, which can be achieved via different, conventional methods.

A first method is asexual (vegetative) propagation. By this approach all plants are derived from a single plant, which, itself, could be the result of a hybrid cross of two distinct parental plants or simply a unique genetic selection.

A second method is seed (sexual) propagation of inbred lines, which produces a uniform plant population. Inbred lines are derived by a process of self-pollination, usually over six or more generations, in order that all the allelic pair of genes on the homologous chromosome pairs are homozygous or identical. The degree of inbreeding (homozygosity) in a line is approached at the rate of 50% per generation, such that by the sixth generation 98.4% purity exist and, by the seventh generation, 99.2%. Thereafter, all plants derived from self-pollination, sibling pollination or random crossing in isolation are essentially genetically identical, and therefore, homozygous and uniform in appearance.

Maximum plant yields, however, do not occur from inbreeding. During the inbreeding process reduction in performance, yield and plant size occur. This reduction in plant vigor through inbreeding is known as “inbreeding depression,” and it is the reason that uniform inbred lines usually are not grown as a commercial crop.

A third method for developing a uniform plant population is hybridization of two inbred lines to produce a uniform, first-generation (F1) hybrid population. Because of hybrid vigor (heterosis), maximum yields as well as uniformity are achieved. As described in detail below, the present invention exploits this method to produce first-generation (F1) J. curcas hybrids and harvested seed.

### SUMMARY

The present description relates “female only” (FO) J. curcas plants that can be used for producing FO Jatropha progeny and hybrid plants derived from plants with the FO trait.

In one aspect, therefore, the present disclosure provides a J. curcas plant characterized by an inflorescence with only female flowers. In a preferred embodiment, substantially all of the inflorescence produced by the J. curcas plant produce only female flowers.

In another aspect, the present disclosure provides methodology for producing hybrid J. curcas seed. Such methodology comprises (1) interplanting, in a plot, first J. curcas plants of a phenotype A with second J. curcas plants of a phenotype B, which differs from phenotype A, such that pollen exchange within the plot is constrained to the first and second plants, and then (2) harvesting fruits from the first plants, whereby F1 hybrid (A×B) seed is obtained from the first plants. In this method, phenotype A comprises the female-only flowering trait and phenotype B does not. Preferably, the plot where the interplanting takes place is located in an area that is substantially free of other Jatropha plants; that is, in a area where the incidence of pollen from Jatropha plants outside of the plot is minimal.

In a further embodiment, the first plants are of a line that is homozygous for the FO trait. Such first plants may have been expanded clonally through vegetative propagation. In another embodiment, the first plants and the second plants are from different inbred lines. Preferably, these lines are genetically distinct such that, when the first and second plants are crossed as described above, the resulting F1 hybrid progeny manifest vigor and seed yield that is superior to that of either parental line.

### BRIEF DESCRIPTION OF THE DRAWINGS

FIG. 1 provides a close-up view of a J. curcas plant with the FO trait; that is, the plant produces inflorescence with female-only flowers.

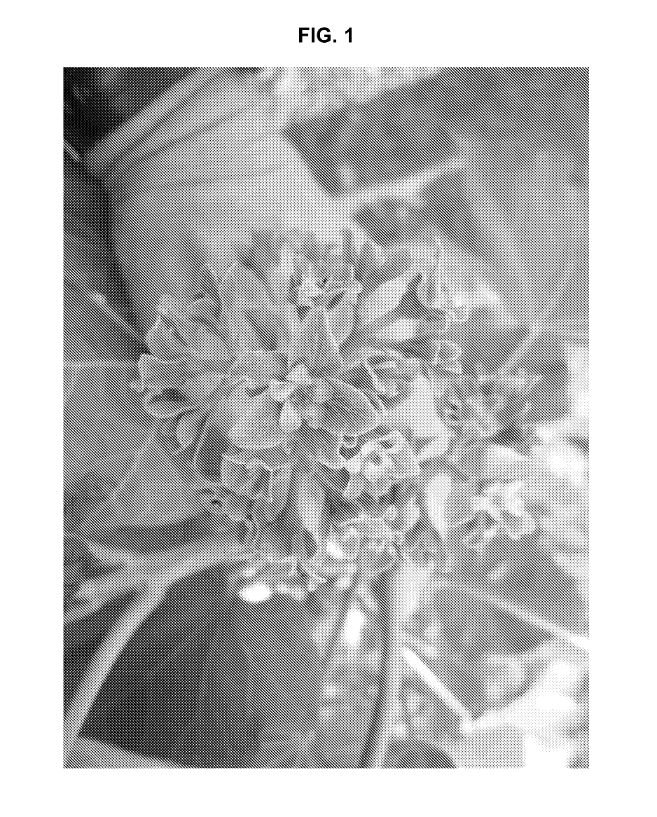

FIG. 2 provides a close-up view of a normal (wild-type) J. curcas plant, which produces inflorescence with both male flowers and female flowers.

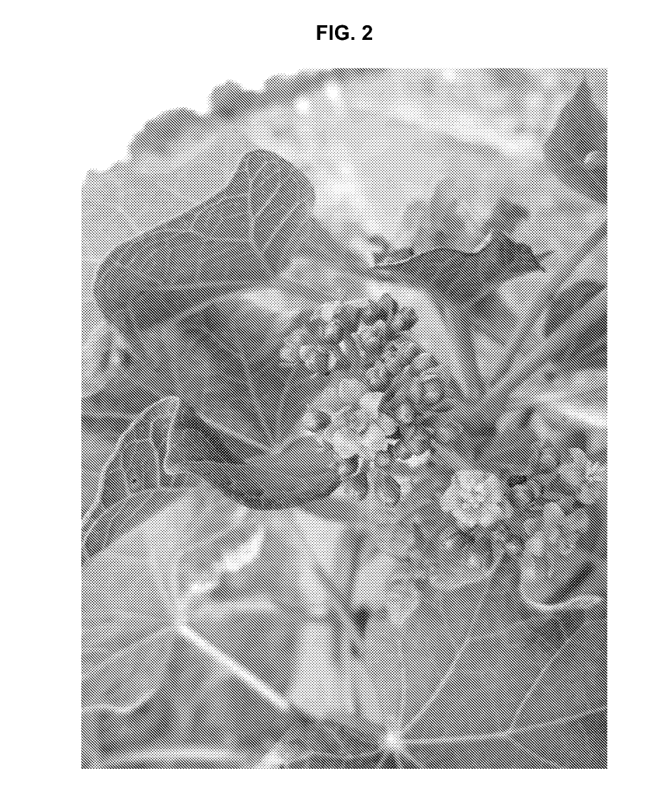

### DETAILED DESCRIPTION

The present disclosure relates the selection and use of a J. curcas phenotype that is characterized by the production of inflorescence with female-only flowers, in contrast to the generally recognized J. curcas phenotype, characterized by inflorescence with both female and male flowers.

This disclosure is unique, too, for its providing the first hybridization paradigm for obtaining commercial scale numbers of uniform F1 Jatropha curcas hybrids via seed propagation. In this regard, hybrid J. curcas seed can be produced by (1) interplanting, in a plot, first J. curcas plants of a phenotype A with second J. curcas plants of a phenotype B, which differs from phenotype A, such that pollen exchange within the plot is constrained to the first and second plants, and then (2) harvesting fruits from the first plants, whereby F1 hybrid (A×B) seed is obtained from the first plants. Phenotype A comprises the female-only flowering trait and phenotype B does not. Preferably, the parental lines are genetically distinct such that, when the first and second plants are crossed as described above, the resulting F1 hybrid progeny manifest vigor and seed yield that is superior to that of either parental line.

Accordingly, the FO J. curcas plants disclosed here can be used in the same way as other J. curcas plants for use in biofuel production or as a living fence. Yet, the FO trait affords the instant J. curcas plants particular advantages over wild-type J. curcas plants. For instance, the FO trait will allow for efficient F1 hybrid seed production at a fraction of the labor and land costs of producing and transporting F1 hybrid plants derived from clonal propagation of shoots. Further, the F1 hybrid plants produced as a result of interplanting would have better root growth as a result of being grown from the harvested F1 hybrid seed as opposed to hybrid plants that are derived from stem cuttings.

The following definitions of terms and phrases employed in this description are provided to illuminate the present invention and to guide the knowledgeable reader in its practice. Unless otherwise noted, the words used here are to be understood according to conventional usage. The description mentions conventional techniques for asexual and sexual reproduction methods.

A cultivar or a variety is a group of similar plants that belong to the same species and that, by structural features and performance, may be distinguished from other varieties within the same species. Two essential characteristics of a variety are identity and reproducibility. Identity is necessary so that the variety may be recognized and distinguished from other varieties within the crop species. The distinguishing features may be morphological characteristics, color markings, physiological functions, disease reaction, or performance, but the FO trait is of particular importance in this context.

Most agricultural varieties are pure for the characteristic or for those characteristics that identify the variety, per se. Reproducibility is needed in order that the characteristic(s) by which the variety is identified will be reproduced in the progeny. Populations that are increased from a single genotype or a mixture of genotypes are referred to as “strains.” Once a strain is identified as superior, it may be named, increased, and made available commercially as a “cultivated variety” or “cultivar.”

In this description, therefore, the terms “cultivar” and “variety” are used synonymously to refer to a group of plants within a species (here, Jatropha curcas) that share certain constant characters, including the FO trait, which separate them from the typical form and from other possible varieties within that species. While possessing at least the distinctive FO trait, a “variety” of the invention also may be characterized by a substantial amount of overall variation between individuals within the variety, based primarily on the Mendelian segregation of traits among the progeny of succeeding generations. On the other hand, “cultivar” or “variety” also can denote a clone, since a Jatropha curcas cultivar may individually be reproduced asexually, via stem cuttings, and all of the clones would be essentially identical genetically.

As distinguished from a “variety,” a “line” is a group of plants that display less variation between individuals, generally (although not exclusively) by virtue of several generations of self-pollination. For purposes of this invention, a “line” is defined sufficiently broadly to include a group of plants vegetatively propagated from a single parent plant, using stem cuttings or tissue culture techniques.

Observed patterns of inheritance of the FO trait are consistent with a single recessive locus that, when homozygous, manifests the FO phenotype or trait. Because FO plants lack male flowers, self-fertilization is impracticable for demonstrating a “true-breeding” state for the trait, i.e., a state where a significant amount of independent segregation of the trait among progeny is not observed. By contrast, plants heterozygous for the FO trait and the wild type produce flowers of both sex and can be self-pollinated. Progeny from such self-pollination will reproduce the FO phenotype in an approximate ratio of 1 FO for every 3 wild type, consistent with the recessive nature of the trait. Similarly, plants manifesting the FO phenotype can be pollinated by sibling plants that, while similar in genetic constitution, are heterozygous for the FO trait. Among the resultant progeny will be plants that segregate 1:1 for the FO trait and plants that are heterozygous for the FO trait and have a wild-type appearance. In this circumstance the FO trait is said to be true-breeding and, if the trait is present in an otherwise genetically uniform background, then progeny will vary predictably with regard to the FO trait, based on Mendelian patterns of inheritance.

Progeny denotes the generation that follows a crossing of parental plants. Progeny in the present invention also can be considered the offspring or descendants of a group of plants.

An inbred line is produced by sibling crossing or self pollination over several generations to produce a genetically homozygous plant selection. A hybrid cultivar is produced by crossing two genetically distinct, inbred lines, collecting seeds produced by the cross, and then germinating seed thus produced to make hybrid plants. The hybrid seeds and plants produced by this method are uniform with respect to their morphological and physiological characteristics. The hybrid seeds produced by this process also benefit from the effect of heterosis (hybrid vigor).

The Female Only (FO) trait arises from a naturally occurring mutation of normal or wild-type Jatropha plants. In the present invention, J. curcas plants comprising the FO trait produce inflorescence with female (stamen-less) flowers but no male flowers, in contrast to the wild-type, monoecious inflorescence of separate male and female flowers within the same inflorescence. Jatropha curcas plants that display the FO trait are effectively male-sterile and can be used, in accordance with this invention, as a parent in hybrid seed production.

The sizeable numbers of FO plants typically needed for hybrid seed production can be obtained by clonal propagation, via cutting or tissue culture, in a conventional manner. Alternatively, selections of seeds or seedlings that are homozygous for the FO trait can be made, using linked molecular markers.

Inflorescence refers to an arrangement of flowers on a stem or axis of the plant. In known J. curcas plants of wild-type habit, inflorescences are formed terminally on branches and produce both male and female flowers. In a FO J. curcas of the present invention, inflorescences are formed terminally on branches and produce only female flowers.

Interplanting denotes a system of planting two or more crops or cultivars in the same field, either mixed together or arranged in separate, alternating rows. Pursuant to this invention, the two parental cultivars are each derived from a single parent via clonal propagation. The system of interplanting is used to allow insects to transport pollen from the normal flower type to the FO flower types, thus producing Jatropha hybrid seed on the FO plants that, upon germination, yield plants that are hybrids of the FO parental line and the normal parental line.

For instance, a 1:2 planting ratio can be employed to this end, although other interplanting configurations might also be utilized. A single row of a wild-type J. curcas inbred or clonally-derived line is planted to border on two successive rows of a FO J. curcas clonal cultivar, as illustrated below:

| | | | | | | | | | |
|---|---|---|---|---|---|---|---|---|---|
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |

In keeping with the invention, different planting schemes also may be employed for efficient plant pollination and hybrid seed production. Illustrative of such other schemes are those that entail, respectively, an alternating between one row of FO and one row of normal types, an alternating between three rows of FO type to one row of normal type, and a staggering the rows with alternating FO and normal types side by side within a row.

Interplanting is an efficient planting system to produce hybrids since one uses only a fraction of the land that is required to produce hybrids by vegetative propagation. For instance, by utilizing an interplanting system, pursuant to the invention, a 10-hectare (ha) production plot, at 2,000 plants per ha yielding 6,000 kg or seed/ha, will produce about 57 million Jatropha hybrid seed per annum. This would be enough seed to plant 28,500 ha in hybrid Jatropha. By vegetative propagation, on the other hand, a 500-ha production plot would be required to produce 57 million hybrid Jatropha cuttings assuming about 60 stem cuttings per plant per year.

Pollen exchange is a process that entails cross-pollination and resulting fertilization of FO inbred J. curcas plants by wild-type inbred J. curcas plants. Pollen exchange typically occurs by means of insects, which carry pollen from the wild-type plants to the FO inbred plants.

Improved Combining Capacity is employed here to denote the capacity to produce hybrids with vigor and yield above that of either the FO parent or a normal or wild-type parent.

### Occurrence of the FO Phenotype

FO Jatropha plants are publicly accessible via seed deposit with an International Deposit Authority. Specifically, FO Jatropha seeds are deposited with the American Culture Type Collection (ATCC), 10801 University Boulevard, Manassas, Va. 20110-2209. 2500 seeds of FO Jatropha were deposited with the ATCC on Dec. 30, 2010 and accorded ATCC Patent Deposit Designation No.: PTA-______. As detailed in Example 3, the deposited seeds can be planted to produce J. curcas plants having inflorescence with FO flowers, and the produced J. curcas plants exhibiting the FO trait can further be crossed with wild-type J. curcas plants to reproducibly and predictably produce J. curcas hybrid seed containing the FO trait according to the methods described here.

Communications with other groups and individuals indicate that the FO trait is observed in other collections of Jatropha curcas obtained from the wilds. Accordingly, it is understood that the FO trait persists in wild populations and, hence, is accessible to the public.

### Pattern of Inheritance of the FO Trait

Crosses of FO plants with normal plants (wild-type inflorescences) yield progeny classes of two types depending on the genotype of the male parent. The first progeny class consists of plants producing only normal inflorescences, and the second class consists of progeny plants that produce a mixture of FO progeny and normal progeny in about a 50-50 ratio.

This pattern of inheritance for the FO trait is consistent with that of a recessive nuclear trait, with some normal plants being heterozygous for the FO allele and normal allele and other normal plants being homozygous for the normal allele. The observed pattern of inheritance observed also is consistent with the FO trait being a type of cytoplasmic male sterility. In that situation the FO types would lack the nuclear restorer allele, some normal types would be heterozygous for a nuclear restorer gene, and other normal types would be homozygous for the nuclear restorer gene.

The following examples further illustrate the invention described above. In relation to these examples, many variations are apparent that yield a like or similar result, without departing from the spirit and scope of the invention. Accordingly, the examples are not limiting of the invention.

### Example 1

### FO Trait Origination and Hybrid Seed Production

Methods for general cultivation and breeding methods of Jatropha cultivars are described in Achten et al., Biomass and Bioenergy 32: 1063-84 (2008).

Jatropha curcas lines that display the above-discussed FO trait were developed in Petén and La Máquina, Guatemala. The object of the breeding program was to develop new J. curcas hybrid plants with increased female flowering, increased fruit and seed count, and increased oil yield.

The inventor selected an unpatented, proprietary accession J. curcas designated ‘Female Only,’ upon observing it in 2009, growing in a controlled breeding improvement program in Petén Guatemala. Jatropha curcas ‘Female Only’ was selected by the inventor based on its whole plant mutation from wild-type J. curcas. In particular, ‘Female Only’ produces inflorescence with only female (stamen-less) flowers and thus, lacks any male flowers.

Only the female flowers of Jatropha plants produce fruit and seed. Thus, the number of female flowers per inflorescence and subsequent number of fruit capsules and seed produced per female flower affects the potential oil yield per Jatropha plant. The inventor recognized the potential of using the FO J. curcas ‘Female Only’ in a breeding program to produce hybrid seeds efficiently from this normally monoecious species.

### Example 2

### FO Hybrid Seed Production

Methods for hybrid crop and seed productions are described in U.S. Pat. No. 4,326,358, U.S. Pat. No. 4,527,352, U.S. Pat. No. 4,627,192, U.S. Pat. No. 4,686,319 and U.S. Pat. No. 6,018,101, as well as in published U.S. application 2008-0098492. The respective contents of these patent publications are incorporated herein by reference.

Hybrid production requires a female line in which no male gametes are engendered. In some instances, the procedure of emasculation is carried out to make a plant devoid of pollen and thus female. Another way to establish a female line for hybrid seed production is to identify a line that is unable to produce viable pollen.

The FO trait of the selected, proprietary accession Jatropha curcas designated ‘Female Only’ was used to create a female line for hybrid seed production, since ‘Female Only’ only produces female flowers and lacks any male flowers. The FO trait of J. curcas ‘Female Only’ allows for hybrid seed production via the female only inbred line being interplanted with a J. curcas inbred line having wild-type flowering, and also having a genetic background that has improved combining capacity with a line bearing only female flowers.

A hybrid seed production plot, for example, with a 1:2 planting ratio can be arranged with a single row of wild-type J. curcas inbred plants planted to border two rows of FO J. curcas inbred plants, in an area that is largely free of other Jatropha plants so that insects pollinating the FO line, only obtain and carry pollen from the wild-type J. curcas inbred line onto the FO J. curcas line, and thus, produce hybrid F1 seed. The distance between each row should be 3 meters and the distance between each plant should also be 2 meters, but other planting densities may also be appropriate. Fruits are then harvested only from the female only (FO) inbred line and the seeds obtained from the FO line are the F1 hybrid seeds. The harvested F1 hybrid seed produce J. curcas plants that exhibit normal flowering, improved vigor, increased fruit and seed count, and increased oil yield. Seeds can also be collected from the wild-type male line for oil processing.

At 6,000 kg/ha/year of seed production, a 10 hectare production plot would yield 60,000 kg of seed, of which 40,000 kg would be F1 hybrid seed as the FO type compose ⅔ of the plants in each ha. At an average seed weight of about 0.7 g/seed this would be about 57 million seeds or, as noted above, enough seed to plant 28,500 hectares with F1 hybrids at a density of 2,000 hybrids per ha. The interplanting process using a selected FO J. curcas line and a wild-type J. curcas line to produce hybrid F1 seed is advantageous, therefore, since the process would involve a fraction of the labor and land costs in comparison to producing a clonally-derived F1 hybrid via vegetative propagation. It also reduces the cost of transporting hybrid plants to a new plantation. Shipping seeds is much less costly than shipping clonally derived cuttings of a hybrid. Clonal propagation of a hybrid via cuttings is more costly to produce, and more costly to ship to prospective customers in comparison to collecting and shipping hybrid seeds.

### Example 3

### Seed Deposit with International Deposit Authority

A seed deposit of a J. curcas selection containing the FO trait were deposited in the American Culture Type Collection (ATCC), 10801 University Boulevard, Manassas, Va. 20110-2209. 2500 seeds of the J. curcas selection containing the FO trait were deposited with the ATCC on Dec. 30, 2010, and accorded ATCC Patent Deposit Designation No. PTA-______. The deposited seeds can be planted to produce J. curcas plants which produce inflorescence with FO flowers, and the produced J. curcas plants exhibiting the FO trait can further be crossed with wild-type J. curcas plants to reproducibly and predictably produce J. curcas hybrid seed containing the FO trait according to the methods described here.

### Example 4

### Genetic Characterization of FO Trait

Asexual propagation of a J. curcas selection containing the FO trait by vegetative cutting was performed in February of 2009 in Petén, Guatemala. First-year evaluation demonstrated that the FO trait is fixed and retained through the first generation of asexual reproduction, and this result should pertain through successive generations of asexual reproduction.

Good concordance was observed in November of 2009 between breeding trials in both Petén and La Máquina, Guatemala, in the percentage of FO types within a given J. curcas accession family, supporting that the FO trait is controlled by genetic rather than environmental factors. The number of female only types within an accession family was either about ¼ or 1/2, which suggests the FO trait may be recessive in nature. The ¼ segregation could arise where seeds come from self-pollination of wild-type J. curcas flowers that are heterozygous for the FO trait, and the ½ segregation could arise when female-only J. curcas flowers receive pollen from neighboring wild-type Jatropha flowers that are heterozygous for the FO trait.

Regardless of what the genetic basis for control of the FO trait in J. curcas may be, these data demonstrate that the FO trait can be reproducibly and predictably introgressed into J. curcas genetic backgrounds and maintained stably through clonal propagation.

### Example 5

### Characteristics of J. curcas Displaying the FO Trait

The FO type J. curcas grows in the same locations and under the same environmental conditions as do normal J. curcas plants.

Open pollinated Jatropha curcas seed were first germinated under typical greenhouse conditions, and development was arrested once seedlings (with shoots) were about 8 to 10 inches in length. Jatropha curcas seedlings then were planted in a field nursery in Petén, Guatemala, under drip irrigation and plastic mulch weed control.

Approximately 6,000 Jatropha curcas open pollinated seedlings were first planted in late 2008 and early 2009 in a nursery in Antigua, Guatemala, and subsequently transplanted to the field in Petén, Guatemala in May of 2009. Systematic screens of the J. curcas plants were conducted to identify traits of potential value, and the FO trait was identified within this population as unique in producing flowers that were all female

### Example 6

### Exemplary Uses of the FO Trait in Other Jatropha Varieties

The FO J. curcas plants of the present invention can be used in the same way as other J. curcas plants for use in biofuel production or as a living fence. However, the FO trait affords the J. curcas plants of the present invention particular advantages over wild-type J. curcas plants. For instance, the FO trait will allow for efficient F1 hybrid seed production at a fraction of the labor and land costs of producing and transporting F1 hybrid plants derived from clonal propagation of shoots. Further, the F1 hybrid plants produced as a result of interplanting would have better root growth as a result of being grown from the harvested F1 hybrid seed as opposed to hybrid plants that are derived from stem cuttings.

### Example 7

### Introduction of FO Trait into Other Jatropha Varieties

The morphological and physiological characteristics, including the FO trait, of the J. curcas plants of the present invention can be introduced into other Jatropha varieties by conventional breeding techniques.

For instance, FO J. curcas plants of the invention can be grown in pollination proximity to another variety of wild-type Jatropha, allowing for manual cross-pollination to be performed between the FO type and the wild-type, and then harvesting the hybrid seeds.

Plants grown from these hybrid seeds can then be visually screened/tested for the maintenance of the FO trait, or self pollinated and the F2 progeny screened for the FO trait and other desirable traits coming from the wild-type Jatropha.

Pursuant to the invention, therefore, clones of plants bearing the FO trait were planted in alternating rows (12-15 plants per row) with normal plants (12-15 plants per row) as a pollen source, thereby to produce normal amounts of fruits and hybrid seeds via insect-mediated hybridization. In addition, the planting density of FO plants was varied by planting two rows of FO clones for every row of normal plants as a pollen source. In each case, total seed production per 7 month-old FO clone (an average of 25 fruits per plant yielding a total of 3,703 seeds) was similar to or exceeded that obtained during the same year in the same general area with 7 month-old FO clones that were hand pollinated.

These results demonstrate that the FO trait can be used in large-scale plantings that are intermixed with combining males to produce hybrid seed via insect-mediated hybridization. By this approach, according to the invention, seeds collected from rows of FO plants will be hybrid seed. Even greater ratios of FO plants per combining male (i.e., 3 rows of FO//1 row of normal or 4 rows of FO//1 row of normal) could be used for even more efficient production of hybrid seeds per unit area, pursuant to the invention.

The provision of the FO J. curcas plants of the present invention enables the production of FO Jatropha progeny and hybrid plants derived from the FO trait. As noted above, the “progeny” category includes plants that are the offspring or descendants of any FO J. curcas plants of the present invention. “Progeny” also includes successive generations of the offspring, for example, those plants selected for the FO trait via methodology described here. First-generation progeny may retain the FO trait of the FO J. curcas parent. Yet, if F1 progeny do not manifest the FO phenotype, then a portion of the subsequent generations of offspring derived from self pollinations will manifest the female-only flowering phenotype and have the same FO trait of the FO J. curcas plants described here.

Any methodology comprising a technique selected from the group consisting of asexual reproduction techniques and sexual reproduction techniques, and using the FO J. curcas plants of the present invention, are contemplated by this invention. Thus, the invention encompasses use of diverse techniques, including but not limited to vegetative propagation, selfing, backcrossing, hybrid production, crossing, and the like, using the FO J. curcas plants of the present invention.

Also within this invention are the progeny or hybrid plants, seed, and plant parts obtained from the FO J. curcas plants of the present invention.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

All publications and patent applications are herein incorporated by reference to the same extent as if each individual publication or patent application were specifically and individually indicated to be incorporated by reference in its entirety.

1-8. (canceled)

9. A method of producing Jatropha curcas oil, comprising pollinating flowers of a first, female only (FO) flowering trait Jatropha curcas plant (phenotype A), with pollen from a second, different Jatropha curcas plant that does not exhibit a female only (FO) flowering trait (phenotype B), to produce a Jatropha curcas F1 hybrid plant, and extracting oil from seeds of said F1 hybrid Jatropha curcas plant.

10. Jatropha curcas oil produced by the method of claim 9.

11. The method of claim 9, further comprising processing said oil for use as a diesel and jet fuel substitute.

12. A diesel and jet fuel substitute produced by the method of claim 11.

13. A method of producing Jatropha curcas F1 hybrid seed, comprising pollinating flowers of a first, female only (FO) flowering trait Jatropha curcas plant (phenotype A), with pollen from a second, different Jatropha curcas plant that does not exhibit a female only (FO) flowering trait (phenotype B), to produce a Jatropha curcas F1 hybrid plant, and harvesting seed from said F1 hybrid Jatropha curcas plant.

14. Jatropha curcas F1 hybrid seed produced by the method of claim 13.

15. A method of producing Jatropha curcas oil, comprising obtaining seeds from a Jatropha curcas F1 hybrid plant, wherein the Jatropha curcas F1 hybrid plant is produced by pollinating flowers of a first, female only (FO) flowering trait Jatropha curcas plant (phenotype A), with pollen from a second, different Jatropha curcas plant that does not exhibit a female only (FO) flowering trait (phenotype B), to produce a Jatropha curcas F1 hybrid plant, and extracting oil from said seeds.

16. The method of claim 15, further comprising processing said oil for use as a diesel and jet fuel substitute.

17. A diesel and jet fuel substitute produced by the method of claim 16.
